(19) United States
(12) Patent Application Publication
(10) Pub. No.: US 2023/0210400 A1
(43) Pub. Date: Jul. 6, 2023

(54) EAR-WEARABLE DEVICES AND METHODS FOR RESPIRATORY CONDITION DETECTION AND MONITORING

(71) Applicant: Starkey Laboratories, Inc., Eden Prairie, MN (US)

(72) Inventors: Krishna Chaithanya Vastare, Eden Prairie, MN (US); Martin McKinney, Minneapolis, MN (US); Nitzan Bornstein, Tel Aviv (IL)

(21) Appl. No.: 18/147,347

(22) Filed: Dec. 28, 2022

Related U.S. Application Data
(60) Provisional application No. 63/295,071, filed on Dec. 30, 2021.

(51) Int. Cl. A61B 5/08; A61B 7/00; A61B 5/00; A61B 5/113

(52) U.S. Cl. CPC A61B 5/0803; A61B 7/003; A61B 5/7264; A61B 5/6815; A61B 5/002; A61B 5/742; A61B 5/1135; A61B 2562/0204

(57) ABSTRACT

Embodiments herein relate to ear-wearable systems and devices that can detect respiratory conditions and related parameters. In an embodiment, an ear-wearable device for respiratory monitoring is included having a control circuit, a microphone, and a sensor package. The ear-wearable device can be configured to analyze signals from the microphone and/or the sensor package and detect a respiratory condition and/or parameter based on analysis of the signals. In an embodiment, an ear-wearable system for respiratory monitoring is included having an accessory device and an ear-wearable device. In an embodiment, a method of detecting respiratory conditions and/or parameters with an ear-wearable device system is included. Other embodiments are also included herein.

This application claims the benefit of U.S. Provisional Application No. 63/295,071 filed Dec. 30, 2021, the content of which is herein incorporated by reference in its entirety.

### FIELD

Embodiments herein relate to ear-wearable systems, devices, and methods. Embodiments herein further relate to ear-wearable systems and devices that can detect respiratory conditions and related parameters.

### BACKGROUND

Respiration includes the exchange of oxygen and carbon dioxide between the atmosphere and cells of the body. Oxygen diffuses from the pulmonary alveoli to the blood and carbon dioxide diffuses from the blood to the alveoli. Oxygen is brought into the lungs during inhalation and carbon dioxide is removed during exhalation.

Generally, adults breathe 12 to 20 times per minute. To start inhalation, the diaphragm contracts, flattening itself downward and enlarging the thoracic cavity. The ribs are pulled up and outward by the intercostal muscles. As the chest expands, the air flows in. For exhalation, the respiratory muscles relax and the chest and thoracic cavity therein returns to its previous size, expelling air from the lungs.

Respiratory assessments, which can include evaluation of respiration rate, respiratory patterns and the like provide important information about a patient's status and clues about necessary treatment steps

### SUMMARY

Embodiments herein relate to ear-wearable systems and devices that can detect respiratory conditions and related parameters. In a first aspect, an ear-wearable device for respiratory monitoring can be included having a control circuit, a microphone, wherein the microphone can be in electrical communication with the control circuit, and a sensor package, wherein the sensor package can be in electrical communication with the control circuit. The ear-wearable device for respiratory monitoring can be configured to analyze signals from the microphone and/or the sensor package and detect a respiratory condition and/or parameter based on analysis of the signals.

In a second aspect, in addition to one or more of the preceding or following aspects, or in the alternative to some aspects, the ear-wearable device for respiratory monitoring can be configured to operate in an onset detection mode and operate in an event classification mode when the onset of an event can be detected.

In a third aspect, in addition to one or more of the preceding or following aspects, or in the alternative to some aspects, the ear-wearable device for respiratory monitoring can be configured to buffer signals from the microphone and/or the sensor package, execute a feature extraction operation, and classify the event when operating in the event classification mode.

In a fourth aspect, in addition to one or more of the preceding or following aspects, or in the alternative to some aspects, the ear-wearable device for respiratory monitoring can be configured to operate in a setup mode prior to operating in the onset detection mode and the event classification mode.

In a fifth aspect, in addition to one or more of the preceding or following aspects, or in the alternative to some aspects, the ear-wearable device for respiratory monitoring can be configured to query a device wearer to take a respiratory action when operating in the setup mode.

In a sixth aspect, in addition to one or more of the preceding or following aspects, or in the alternative to some aspects, the ear-wearable device for respiratory monitoring can be configured to query a device wearer to reproduce a respiratory event when operating in the setup mode.

In a seventh aspect, in addition to one or more of the preceding or following aspects, or in the alternative to some aspects, the ear-wearable device for respiratory monitoring can be configured to receive and execute a machine learning classification model specific for the detection of one or more respiratory conditions.

In an eighth aspect, in addition to one or more of the preceding or following aspects, or in the alternative to some aspects, the ear-wearable device for respiratory monitoring can be configured to receive and execute a machine learning classification model that can be specific for the detection of one or more respiratory conditions that can be selected based on a user input from amongst a set of respiratory conditions.

In a ninth aspect, in addition to one or more of the preceding or following aspects, or in the alternative to some aspects, the ear-wearable device for respiratory monitoring can be configured to send information regarding detected respiratory conditions and/or parameters to an accessory device for presentation to the device wearer.

In a tenth aspect, in addition to one or more of the preceding or following aspects, or in the alternative to some aspects, the respiratory condition and/or parameter can include at least one selected from the group consisting of respiration rate, tidal volume, respiratory minute volume, inspiratory reserve volume, expiratory reserve volume, vital capacity, and inspiratory capacity.

In an eleventh aspect, in addition to one or more of the preceding or following aspects, or in the alternative to some aspects, the respiratory condition and/or parameter can include at least one selected from the group consisting of bradypnea, tachypnea, hyperpnea, an obstructive respiration condition, Kussmaul respiration, Biot respiration, ataxic respiration, and Cheyne-Stokes respiration.

In a twelfth aspect, in addition to one or more of the preceding or following aspects, or in the alternative to some aspects, the ear-wearable device for respiratory monitoring can be configured to detect one or more adventitious sounds.

In a thirteenth aspect, in addition to one or more of the preceding or following aspects, or in the alternative to some aspects, the adventitious sounds can include at least one selected from the group consisting of fine crackles, medium crackles, coarse crackles, wheezing, rhonchi, and pleural friction rub.

In a fourteenth aspect, an ear-wearable system for respiratory monitoring can be included having an accessory device and an ear-wearable device. The accessory device can include a control circuit and a display screen. The ear-wearable device can include a control circuit, a microphone, wherein the microphone can be in electrical communication with the control circuit, and a sensor package, wherein the sensor package can be in electrical communication with the control circuit. The ear-wearable device can be configured to analyze signals from the microphone and/or the sensor package to detect the onset of a respiratory event and buffer signals from the microphone and/or the sensor package after a detected onset, send buffered signal data to the accessory device, and receive an indication of a respiratory condition from the accessory device. The accessory device can be configured to process signal data from the ear-wearable device to detect a respiratory condition.

In a fifteenth aspect, in addition to one or more of the preceding or following aspects, or in the alternative to some aspects, the ear-wearable system for respiratory monitoring can be configured to operate in an onset detection mode and operate in an event classification mode when the onset of an event can be detected.

In a sixteenth aspect, in addition to one or more of the preceding or following aspects, or in the alternative to some aspects, the ear-wearable device can be configured to buffer signals from the microphone and/or the sensor package when operating in the event classification mode.

In a seventeenth aspect, in addition to one or more of the preceding or following aspects, or in the alternative to some aspects, the ear-wearable system for respiratory monitoring can be configured to operate in a setup mode prior to operating in the onset detection mode and the event classification mode.

In an eighteenth aspect, in addition to one or more of the preceding or following aspects, or in the alternative to some aspects, the ear-wearable system for respiratory monitoring can be configured to query a device wearer to take a respiratory action when operating in the setup mode.

In a nineteenth aspect, in addition to one or more of the preceding or following aspects, or in the alternative to some aspects, the ear-wearable system for respiratory monitoring can be configured to query a device wearer to reproduce a respiratory event when operating in the setup mode.

In a twentieth aspect, in addition to one or more of the preceding or following aspects, or in the alternative to some aspects, the ear-wearable system for respiratory monitoring can be configured to receive and execute a machine learning classification model specific for the detection of one or more respiratory conditions.

In a twenty-first aspect, in addition to one or more of the preceding or following aspects, or in the alternative to some aspects, the ear-wearable system for respiratory monitoring can be configured to receive and execute a machine learning classification model that can be specific for the detection of one or more respiratory conditions that can be selected based on a user input from amongst a set of respiratory conditions.

In a twenty-second aspect, in addition to one or more of the preceding or following aspects, or in the alternative to some aspects, the accessory device can be configured to present information regarding detected respiratory conditions and/or parameters to the device wearer.

In a twenty-third aspect, in addition to one or more of the preceding or following aspects, or in the alternative to some aspects, the respiratory condition can include at least one selected from the group consisting of bradypnea, tachypnea, hyperpnea, an obstructive respiration condition, Kussmaul respiration, Biot respiration, ataxic respiration, and Cheyne-Stokes respiration.

In a twenty-fourth aspect, in addition to one or more of the preceding or following aspects, or in the alternative to some aspects, the ear-wearable system for respiratory monitoring can be configured to detect one or more adventitious sounds.

In a twenty-fifth aspect, in addition to one or more of the preceding or following aspects, or in the alternative to some aspects, the adventitious sounds can include at least one selected from the group consisting of fine crackles, medium crackles, coarse crackles, wheezing, rhonchi, and pleural friction rub.

In a twenty-sixth aspect, a method of detecting respiratory conditions and/or parameters with an ear-wearable device can be included. The method can include analyzing signals from a microphone and/or a sensor package and detecting a respiratory condition and/or parameter based on analysis of the signals.

In a twenty-seventh aspect, in addition to one or more of the preceding or following aspects, or in the alternative to some aspects, the method further can include operating the ear-wearable device in an onset detection mode and operating the ear-wearable device in an event classification mode when the onset of an event can be detected.

In a twenty-eighth aspect, in addition to one or more of the preceding or following aspects, or in the alternative to some aspects, the method can further include buffering signals from the microphone and/or the sensor package, executing a feature extraction operation, and classifying the event when operating in the event classification mode.

In a twenty-ninth aspect, in addition to one or more of the preceding or following aspects, or in the alternative to some aspects, the method can further include operating in a setup mode prior to operating in the onset detection mode and the event classification mode.

In a thirtieth aspect, in addition to one or more of the preceding or following aspects, or in the alternative to some aspects, the method can further include querying a device wearer to take a respiratory action when operating in the setup mode.

In a thirty-first aspect, in addition to one or more of the preceding or following aspects, or in the alternative to some aspects, the method can further include querying a device wearer to reproduce a respiratory event when operating in the setup mode.

In a thirty-second aspect, in addition to one or more of the preceding or following aspects, or in the alternative to some aspects, the method can further include receiving and executing a machine learning classification model specific for the detection of one or more respiratory conditions.

In a thirty-third aspect, in addition to one or more of the preceding or following aspects, or in the alternative to some aspects, the method can further include receiving and executing a machine learning classification model that can be specific for the detection of one or more respiratory conditions that can be selected based on a user input from amongst a set of respiratory conditions.

In a thirty-fourth aspect, in addition to one or more of the preceding or following aspects, or in the alternative to some aspects, the method can further include sending information regarding detected respiratory conditions and/or parameters to an accessory device for presentation to the device wearer.

In a thirty-fifth aspect, in addition to one or more of the preceding or following aspects, or in the alternative to some aspects, the method can further include detecting one or more adventitious sounds.

In a thirty-sixth aspect, in addition to one or more of the preceding or following aspects, or in the alternative to some aspects, the adventitious sounds can include at least one selected from the group consisting of fine crackles, medium crackles, coarse crackles, wheezing, rhonchi, and pleural friction rub.

In a thirty-seventh aspect, a method of detecting respiratory conditions and/or parameters with an ear-wearable device system can be included, the method including analyzing signals from a microphone and/or a sensor package with an ear-wearable device, detecting the onset of a respiratory event with the ear-wearable device, buffering signals from the microphone and/or the sensor package after a detected onset, sending buffered signal data from the ear-wearable device to an accessory device, processing signal data from the ear-wearable device with the accessory device to detect a respiratory condition, and sending an indication of a respiratory condition from the accessory device to the ear-wearable device.

In a thirty-eighth aspect, in addition to one or more of the preceding or following aspects, or in the alternative to some aspects, the method further can include operating in an onset detection mode and operating in an event classification mode when the onset of an event can be detected.

In a thirty-ninth aspect, in addition to one or more of the preceding or following aspects, or in the alternative to some aspects, the method can further include buffering signals from the microphone and/or the sensor package, executing a feature extraction operation, and classifying the event when operating in the event classification mode.

In a fortieth aspect, in addition to one or more of the preceding or following aspects, or in the alternative to some aspects, the method can further include operating in a setup mode prior to operating in the onset detection mode and the event classification mode.

In a forty-first aspect, in addition to one or more of the preceding or following aspects, or in the alternative to some aspects, the method can further include querying a device wearer to take a respiratory action when operating in the setup mode.

In a forty-second aspect, in addition to one or more of the preceding or following aspects, or in the alternative to some aspects, the method can further include querying a device wearer to reproduce a respiratory event when operating in the setup mode.

In a forty-third aspect, in addition to one or more of the preceding or following aspects, or in the alternative to some aspects, the method can further include receiving and executing a machine learning classification model specific for the detection of one or more respiratory conditions.

In a forty-fourth aspect, in addition to one or more of the preceding or following aspects, or in the alternative to some aspects, the method can further include receiving and executing a machine learning classification model that can be specific for the detection of one or more respiratory conditions that can be selected based on a user input from amongst a set of respiratory conditions.

In a forty-fifth aspect, in addition to one or more of the preceding or following aspects, or in the alternative to some aspects, the method can further include presenting information regarding detected respiratory conditions and/or parameters to the device wearer.

In a forty-sixth aspect, in addition to one or more of the preceding or following aspects, or in the alternative to some aspects, the method can further include detecting one or more adventitious sounds.

In a forty-seventh aspect, in addition to one or more of the preceding or following aspects, or in the alternative to some aspects, the adventitious sounds can include at least one selected from the group consisting of fine crackles, medium crackles, coarse crackles, wheezing, rhonchi, and pleural friction rub.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE FIGURES

Aspects may be more completely understood in connection with the following figures (FIGS.), in which:

FIG. 1 is a schematic view of an ear-wearable device and a device wearer in accordance with various embodiments herein.

FIG. 2 is a series of charts illustrating respiratory patterns in accordance with various embodiments herein.

FIG. 3 is a series of charts illustrating respiratory patterns in accordance with various embodiments herein.

FIG. 4 is a schematic view of an ear-wearable device in accordance with various embodiments herein.

FIG. 5 is a schematic view of an ear-wearable device within the ear in accordance with various embodiments herein.

FIG. 6 is a flowchart of operations in accordance with various embodiments herein.

FIG. 7 is a flowchart of operations in accordance with various embodiments herein.

FIG. 8 is a schematic view of an ear-wearable device system in accordance with various embodiments herein.

FIG. 9 is a block diagram view of components of an ear-wearable device in accordance with various embodiments herein.

FIG. 10 is a block diagram view of components of an accessory device in accordance with various embodiments herein.

While embodiments are susceptible to various modifications and alternative forms, specifics thereof have been shown by way of example and drawings, and will be described in detail. It should be understood, however, that the scope herein is not limited to the particular aspects described. On the contrary, the intention is to cover modifications, equivalents, and alternatives falling within the spirit and scope herein.

### DETAILED DESCRIPTION

As discussed above, assessment of respiratory function is an important part of assessing an individual's overall health status. The passage of air into the lungs and back out again creates detectable sound and the movement of the chest and associated muscles creates detectable motion.

In various embodiments, the devices herein incorporate built-in sensors for measuring and analyzing multiple types of signals and/or data to detect respiration and respiration patterns, including, but not limited to, microphone data and motion sensor data amongst others. Data from these sensors can be processed by devices and systems herein to accurately detect the respiration of device wearers.

Machine learning models can be utilized herein for detecting respiration and can be developed and trained with device wearer/patient data, and deployed for on-device monitoring, classification, and communication, taking advantage of the fact that such ear-wearable devices will be continuously worn by the user, particularly in the case of users with hearing-impairment. Further, recognizing that aspects of respiration such as the specific sounds occurring vary from person-to-person embodiments herein can include an architecture for personalization via on-device in-situ training and optimization phase(s).

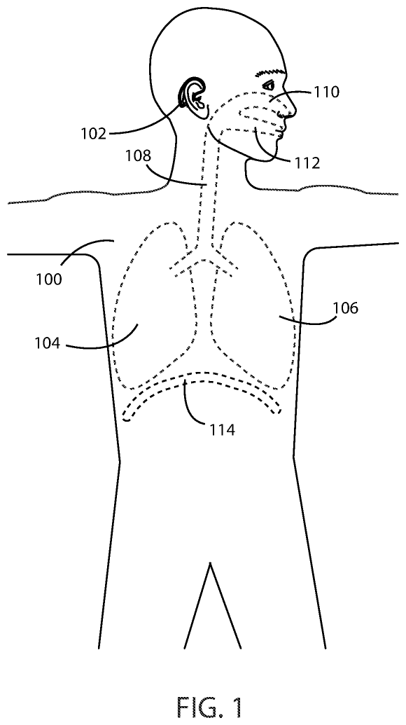

Referring now to FIG. 1, a device wearer 100 is shown wearing an ear-wearable device 102, such as an ear-wearable device for respiratory monitoring. Portions of the anatomy of the device wearer 100 involved in respiration are also shown in FIG. 1. In specific, FIG. 1 shows lungs 104, 106 along with the trachea 108 (or windpipe). The trachea 108 is in fluid communication with the nasal passage 110 and the mouth 112.

FIG. 1 also shows the diaphragm 114. To start inhalation, the diaphragm 114 contracts, flattening itself downward and enlarging the thoracic cavity. The ribs are pulled up and outward by the intercostal muscles. As the chest expands, the air flows in through either the nasal passage 110 or the mouth 112 then passing through the trachea 108 and into the lungs 104, 106. For exhalation, the respiratory muscles relax and the chest and thoracic cavity therein returns to its previous size, expelling air from the lungs 104, 106 through the trachea 108 and back out the nasal passage 110 or the mouth 112. The ear-wearable device 102 can include sensors as described herein that can detect sounds and movement, amongst other things, associated with inhalation and exhalation to monitor respiratory function and/or detect a respiratory condition or parameter.

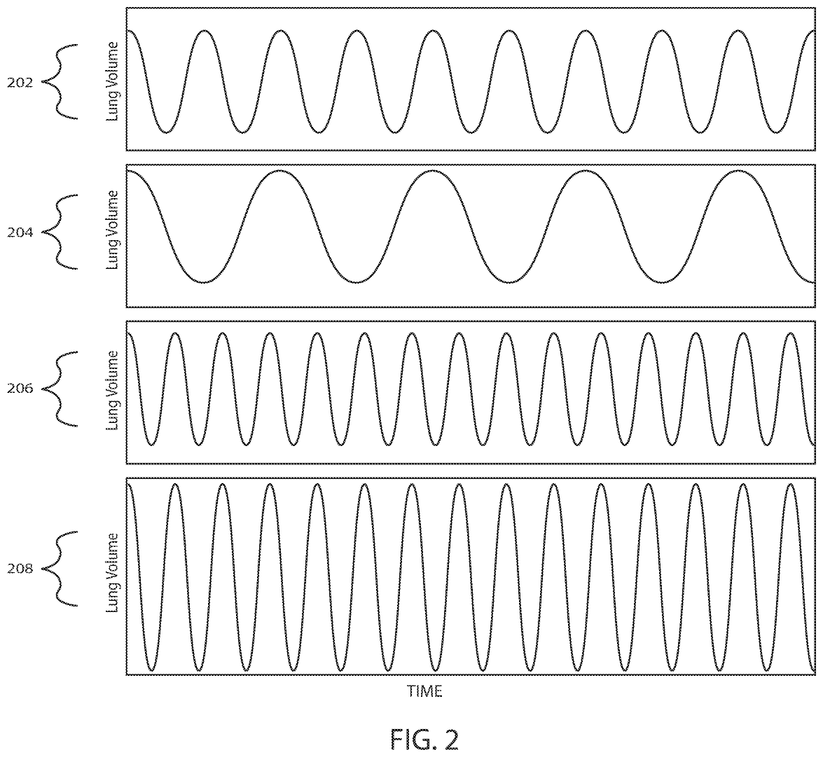

Many different respiratory patterns can be detected with ear-wearable devices and systems herein. Referring now to FIG. 2, charts are shown of lung volume over time demonstrating various respiratory patterns. For example, chart 202 illustrates a normal respiration pattern. Chart 204 illustrates bradypnea or a slower than normal breathing pattern. Bradypnea can include breathing at a rate of less than 12 cycles (inhalation and exhalation) per minute for an adult. Chart 206 illustrates tachypnea or a faster than normal breathing pattern. Tachypnea can include breathing at a rate of greater than 20 cycles per minute for an adult. Chart 208 illustrates hyperpnea sometimes known as hyperventilation. Hyperpnea can include breathing at a rate of greater than 20 cycles per minute for an adult with a greater than normal volume (deep breaths).

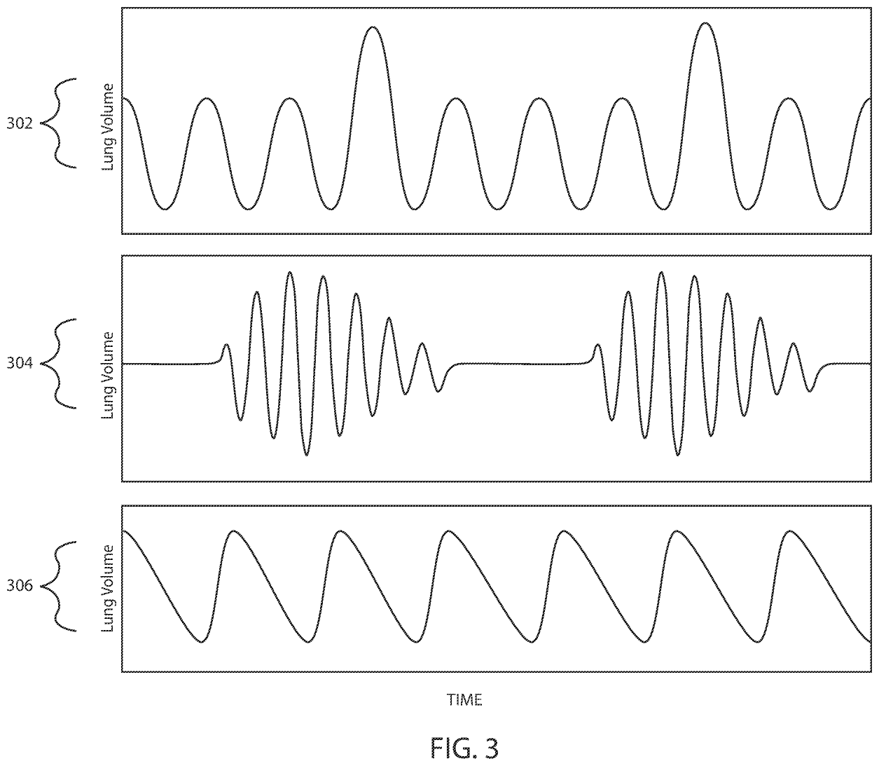

FIG. 3 shows additional charts are shown of lung volume over time demonstrating various respiratory patterns. Chart 302 illustrates a sighing pattern or frequently interspersed deep breathes. Chart 304 illustrates a pattern known as Cheyne-Stokes respiration. Cheyne-Stokes can include periods of fast, shallow breathing followed by slow, heavier breathing and then apneas (moments without any breath at all). Chart 306 illustrates an obstructive breathing pattern where exhalation takes longer than inhalation. These patterns along with many others (such as Kussmaul respiration, Biot respiration, and ataxic breathing patterns) can be detected using ear-wearable devices and systems herein.

Beyond respiratory patterns, devices or systems herein can also identify specific sounds associated with breathing having significance for determining the health status of a device wearer. For example, devices or systems herein can identify adventitious sounds such as fine crackles, medium crackles, coarse crackles, wheezing, rhonchi, pleural friction rub, and the like. Fine crackles refer to fine, high-pitched crackling and popping noises heard during the end of inspiration. Medium crackles refer to medium-pitched, moist sound hear about halfway through inspiration. Coarse crackles refer to low-pitched, bubbling or gurgling sounds that start early in inspiration and extend in the first part of expiration. Wheezing refers to high-pitched, musical sound similar to a squeak which is heard more commonly during expiration, but may also be hear during inspiration. Rhonchi refers to low-pitched, coarse, load, low snoring or moaning tones heard primarily during expiration. Pleural friction rub refers to a superficial, low-pitched coarse rubbing or grating sound like two surfaces rubbing together and can be heard throughout inspiration and expiration.

In various embodiments, various respiration parameters can be calculated and/or estimated by the device or system. By way of example, one or more of respiration rate, tidal volume, respiratory minute volume, inspiratory reserve volume, expiratory reserve volume, vital capacity, and inspiratory capacity can be calculated and/or estimated. In some embodiments, parameters related to volume can be estimated based on a combination of time and estimated flow rate. Flow rate can be estimated based on pitch, where higher flow rates generate higher pitches. A baseline flow rate value can be established during a configuration or learning phase and the baseline flow rate can be associated with a particular pitch for a given individual. Then observed changes in pitch can be used to estimate current flow rates for that individual. It will be appreciated, however, that various techniques can be used to estimate volumes and/or flow rates.

Ear-wearable devices herein, including hearing aids and hearables (e.g., wearable earphones), can include an enclosure, such as a housing or shell, within which internal components are disposed. Components of an ear-wearable device herein can include a control circuit, digital signal processor (DSP), memory (such as non-volatile memory), power management circuitry, a data communications bus, one or more communication devices (e.g., a radio, a near-field magnetic induction device), one or more antennas, one or more microphones (such as a microphone facing the ambient environment and/or an inward-facing microphone), a receiver/speaker, a telecoil, and various sensors as described in greater detail below. More advanced ear-wearable devices can incorporate a long-range communication device, such as a BLUETOOTH® transceiver or other type of radio frequency (RF) transceiver.

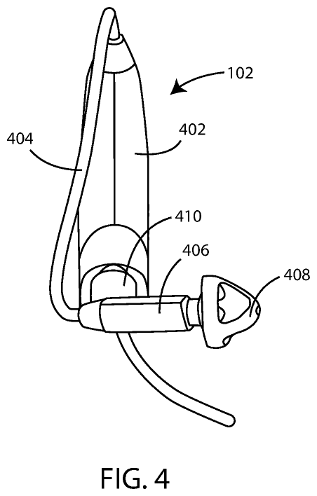

Referring now to FIG. 4, a schematic view of an ear-wearable device 102 is shown in accordance with various embodiments herein. The ear-wearable device 102 can include a device housing 402. The device housing 402 can define a battery compartment 410 into which a battery can be disposed to provide power to the device. The ear-wearable device 102 can also include a receiver 406 adjacent to an earbud 408. The receiver 406 an include a component that converts electrical impulses into sound, such as an electroacoustic transducer, speaker, or loudspeaker. A cable 404 or connecting wire can include one or more electrical conductors and provide electrical communication between components inside of the device housing 402 and components inside of the receiver 406.

The ear-wearable device 102 shown in FIG. 4 is a receiver-in-canal type device and thus the receiver is designed to be placed within the ear canal. However, it will be appreciated that many different form factors for ear-wearable devices are contemplated herein. As such, ear-wearable devices herein can include, but are not limited to, behind-the-ear (BTE), in-the ear (ITE), in-the-canal (ITC), invisible-in-canal (IIC), receiver-in-canal (RIC), receiver in-the-ear (RITE), completely-in-the-canal (CIC) type hearing assistance devices, a personal sound amplifier, implantable hearing devices (such as a cochlear implant, a brainstem implant, or an auditory nerve implant), a bone-anchored or otherwise osseo-integrated hearing device, or the like.

While FIG. 4 shows a single ear-wearable device, it will be appreciated that in various examples, a pair of ear-wearable devices can be included and can work as a system, e.g., an individual may wear a first device on one ear, and a second device on the other ear. In some examples, the same type(s) of sensor(s) may be present in each device, allowing for comparison of left and right data for data verification (e.g., increase sensitivity and specificity through redundancy), or differentiation based on physiologic location (e.g., physiologic signal may be different in one location from the other location.)

Ear-wearable devices of the present disclosure can incorporate an antenna arrangement coupled to a high-frequency radio, such as a 2.4 GHz radio. The radio can conform to an IEEE 802.11 (e.g., WIFI®) or BLUETOOTH® (e.g., BLE, BLUETOOTH® 4.2 or 5.0) specification, for example. It is understood that ear-wearable devices of the present disclosure can employ other radios, such as a 900 MHz radio. Ear-wearable devices of the present disclosure can be configured to receive streaming audio (e.g., digital audio data or files) from an electronic or digital source. Representative electronic/digital sources (also referred to herein as accessory devices) include an assistive listening system, a TV streamer, a remote microphone device, a radio, a smartphone, a cell phone/entertainment device (CPED), a programming device, or other electronic device that serves as a source of digital audio data or files.

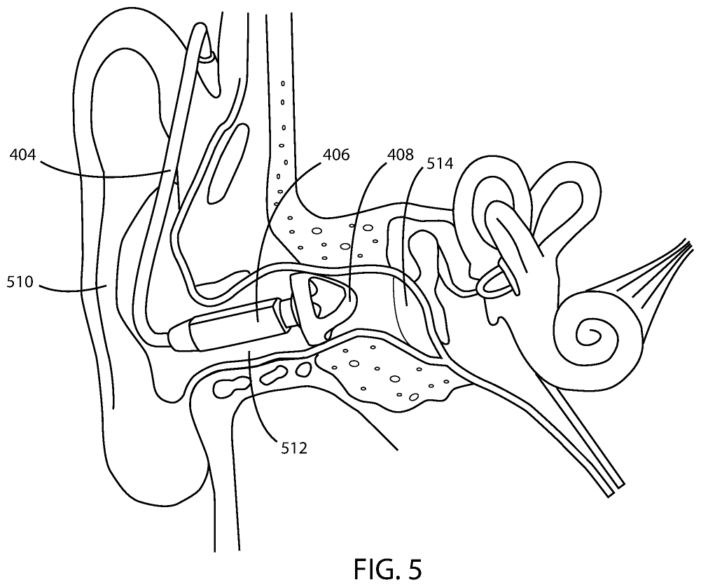

As mentioned above, the ear-wearable device 102 can be a receiver-in-canal (RIC) type device and thus the receiver is designed to be placed within the ear canal. Referring now to FIG. 5, a schematic view is shown of an ear-wearable device disposed within the ear of a subject in accordance with various embodiments herein. In this view, the receiver 406 and the earbud 408 are both within the ear canal 512, but do not directly contact the tympanic membrane 514. The hearing device housing is mostly obscured in this view behind the pinna 510, but it can be seen that the cable 404 passes over the top of the pinna 510 and down to the entrance to the ear canal 512.

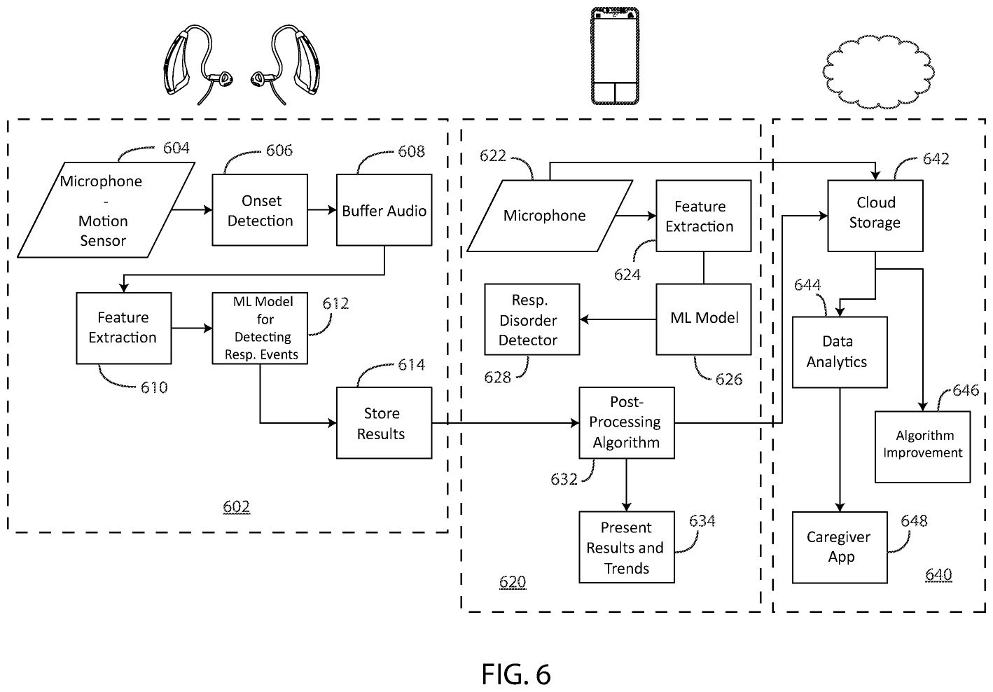

Referring now to FIG. 6, a flowchart is shown of various operations executed in accordance with embodiments herein. Data/signals can be gathered 604 from various sensors including, as a specific example, from a microphone and a motion sensor. These signals can be evaluated 606 in order to detect the possible onset of a respiratory event. Onset can be detected in various ways. In some embodiments, an onset detection algorithm herein detects any event that could be a respiratory disorder. In some embodiments, the onset detection algorithm detects any change in a respiratory parameter (rate, volume, etc.) over a baseline value for the device wearer. Baseline values can be established during a setup mode or phase of operation. In various embodiments, the onset detection algorithm does not actually determine the respiratory pattern or event, rather it just detects the start of respiratory parameters that may be abnormal for the device wearer. In some embodiments, the device wearer can provide an input, such as a button press or a voice command, to bypass the onset detection mode and start analyzing signals/data for respiratory patterns, events, etc.

If the onset of a respiratory event is detected or bypassed via an input from the device wearer, then the ear-wearable devices can buffer 608 signals/data, such as buffering audio data and/or motion sensor data. Buffering can include buffering 0.2, 0.5, 1, 2, 3, 4, 5, 10, 20, 30 seconds worth of signals/data or more, or an amount falling within a range between any of the foregoing. In some embodiments, a sampling rate of sensors and/or a microphone can also be changed upon the detection of the onset of a respiratory event. For example, the sampling rate of various sensors can be increased to provide a richer data set to more accurately detect respiratory events, conditions, patterns, and/or parameters. By way of example, in some embodiments, a sampling rate of a microphone or sensor herein can be increased to at least about 1 kHz, 2 kHz, 3 kHz, 5 kHz, 7 kHz, 10 kHz, 15 kHz, 20 kHz, 30 kHz or higher, or a sampling rate falling within a range between any of the foregoing.

In various embodiments, the ear-wearable device(s) can then undertake an operation of feature extraction 610. Further details of feature extraction are provided in greater detail below. Next, in various embodiments, the ear-wearable device(s) can execute a machine-learning model for detecting respiratory events 612. Then the ear-wearable device(s) can store results 614. In various embodiments, operations 604 through 614 can be executed at the level of the ear-wearable device(s) 602.

In some embodiments, microphone and/or other sensor data can also be gathered 622 at the level of an accessory device 620. In some embodiments, such data can be sent to the cloud or through another data network to be stored 642. In some embodiments, such data can also be put through an operation of feature extraction 624. After feature extraction 624, then the extracted portions of the data can be processed with a machine learning model 626 to detect respiratory patterns, conditions, events, sounds, and the like. In various embodiments, if a particular pattern, event, condition, or sound is detected at the level of the accessory device 620, it can be confirmed back to the ear-wearable device and results can be stored in the accessory device 620 and later to the cloud 640.

In various embodiments, the machine learning model 626 on the accessory device 620 can be a more complex machine learning model/algorithm than that executed on the ear-wearable devices 602. In some embodiments, the machine learning model/algorithm that is executed on the accessory device 620 and/or on the ear-wearable device(s) 602 can be one that is optimized for speed and/or storage and execution at the edge such as a TensorFlow Lite model.

Based on the output of applying the machine learning model 626, various respiratory conditions, disorders, parameters, and the like can be detected 628.

In some embodiments, results generated by the ear-wearable device can be passed to an accessory device and a post-processing operation 632 can be applied. In some embodiments, the device or system can present information 634, such as results and/or trends or other aspects of respiration, to the device wearer or another individual through the accessory device. In some embodiments, the results from the ear-wearable device(s) can be periodically retrieved by the accessory device 620 for presenting the results to the device wearer and/or storing them in the cloud.

In some embodiments, data can then be passed to the cloud or another data network for storage 642 after the post-processing operation 632.

In some embodiments, various data analytics operations 644 can be performed in the cloud 640 and/or by remote servers (real or virtual). In some embodiments, outputs from the data analytics operation 644 can then be passed to a caregiver application 648 or to another system or device. In various embodiments, various other operations can also be executed. For example, in some embodiments, one or more algorithm improvement operations 646 can be performed, such as to improve the machine learning model being applied to detect respiratory events, disorders, conditions, etc.

While not illustrated with respect to FIG. 6, in some embodiments the device and/or system can include operating in a setup mode. In some embodiments, the ear-wearable device can be configured to query a device wearer to take a respiratory action when operating in the setup mode. In this way, the device can obtain a positive example for a particular type of respiratory action or event that can be used with machine learning operations as described in greater detail below. In some embodiments, the ear-wearable device for respiratory monitoring can be configured to query a device wearer to reproduce a particular respiratory event when operating in the setup mode.

It will be appreciated that processing resources, memory, and/or power on ear-wearable devices is not unlimited. Further executing machine-learning models can be resource intensive. As such, in some embodiments, it can be efficient to only execute certain models on the ear-wearable devices. In some embodiments, the device or system can query a system user (which could be the device wearer or another individual such as a care provider) to determine which respiration patterns or sounds are of interest for possible detection. After receiving input regarding respiration patterns or sounds of interest, then only the machine-learning models of relevance for those respiration patterns or sounds can be loaded onto the ear-wearable device. Alternatively, many models may be loaded onto the ear-wearable device, but only a subset may be executed saving processing and/or power resources.

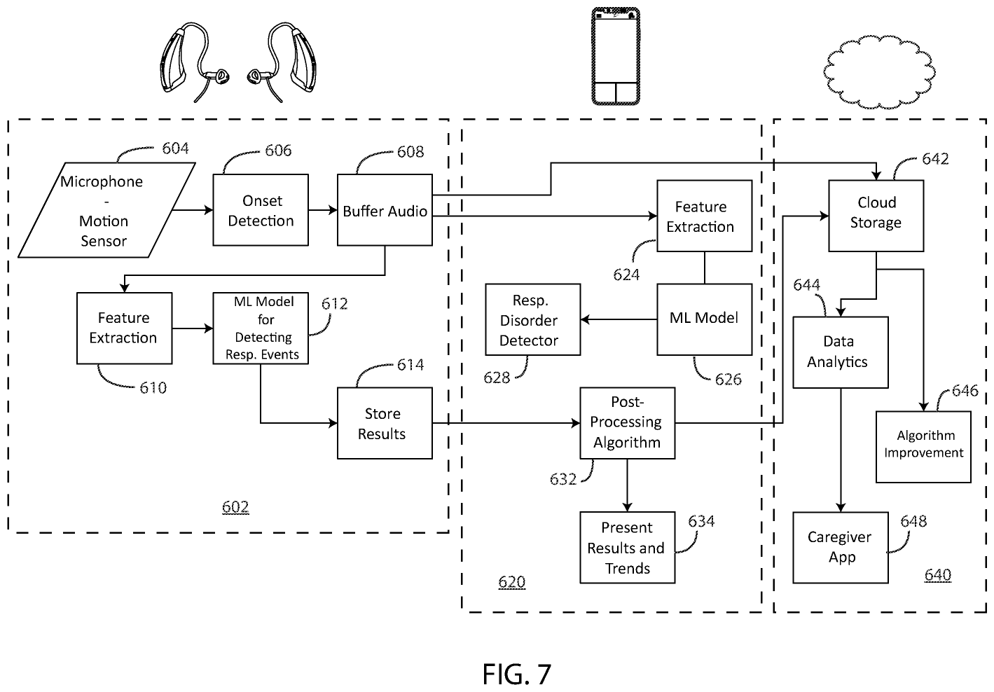

Many different variations on the operations described with respect to FIG. 6 herein are contemplated. Referring now to FIG. 7, another flowchart is shown of various operations executed in accordance with embodiments herein. FIG. 7 is largely similar to FIG. 6. However, in FIG. 7, buffered data such as buffered audio data is passed directly along to the cloud 640, thus bypassing some amount of operations being executed at the level of the accessory device 620.

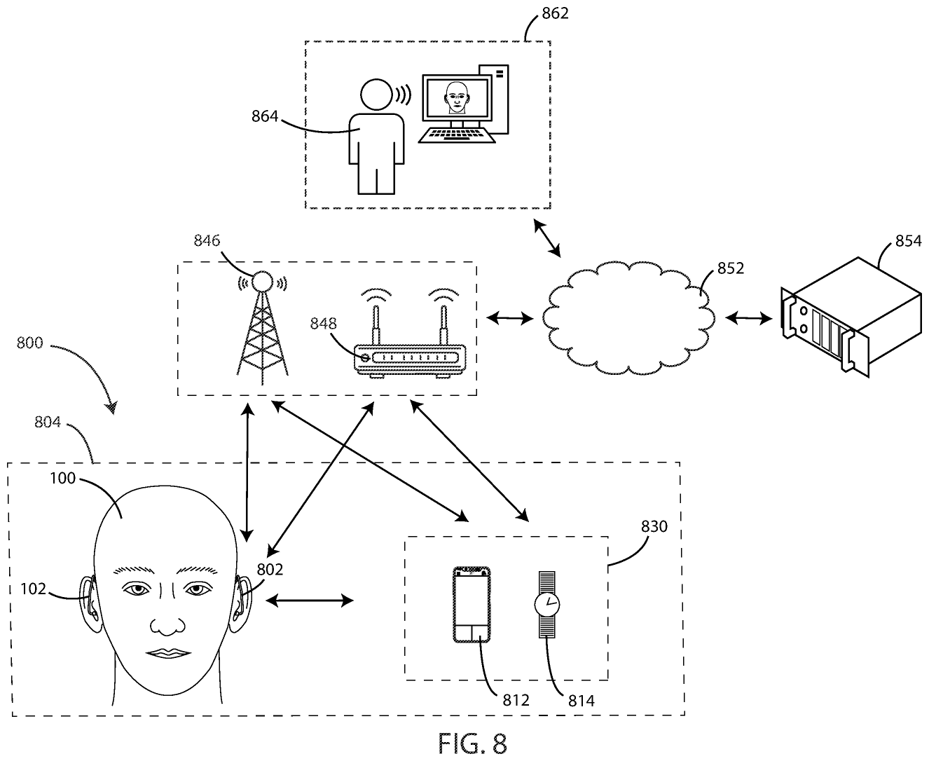

Referring now to FIG. 8, a schematic view of an ear-wearable system 800 is shown in accordance with various embodiments herein. FIG. 8 shows a device wearer 100 with an ear-wearable device 102 and a second ear-wearable device 802. The device wearer 100 is at a first location or device wearer location 804. The system can include and/or can interface with other devices 830 at the first location 804. The other devices 830 in this example can include an external device or accessory device 812, which could be a smart phone or similar mobile communication/computing device in some embodiments. The other devices 830 in this example can also include a wearable device 814, which could be an external wearable device 814 such as a smart watch or the like.

FIG. 8 also shows communication equipment including a cell tower 846 and a network router 848. FIG. 8 also schematically depicts the cloud 852 or similar data communication network. FIG. 8 also depicts a cloud computing resource 854. The communication equipment can provide data communication capabilities between the ear-wearable devices 102, 802 and other components of the system and/or components such as the cloud 852 and cloud resources such as a cloud computing resource 854. In some embodiments, the cloud 852 and/or resources thereof can host an electronic medical records system. In some embodiments, the cloud 852 can provide a link to an electronic medical records system. In various embodiments, the ear-wearable system 800 can be configured to send information regarding respiration, respiration patterns, respiration events, and/or respiration conditions to an electronic medical record system.

In some embodiments, the ear-wearable system 800 can be configured to receive information regarding respiration as relevant to the individual through an electronic medical record system. Such received information can be used alongside data from microphones and other sensors herein and/or incorporated into machine learning classification models used herein.

FIG. 8 also shows a remote location 862. The remote location 862 can be the site of a third party 864, which can be a clinician, care provider, loved one, or the like. The third party 864 can receive reports regarding respiration of the device wearer. In some embodiments, the third party 864 can provide instructions for the device wearer regarding actions to take. In some embodiments, the system can send information and/or reports to the third party 864 regarding the device wearer's condition and/or respiration including trends and/or changes in the same. In some scenarios, information and/or reports can be sent to the third party 864 in real-time. In other scenarios, information and/or reports can be sent to the third party 864 periodically.

In some embodiments, the ear-wearable device and/or system herein can be configured to issue a notice regarding respiration of a device wearer to a third party. In some cases, if the detected respiration pattern is indicative of danger to the device wearer, emergency services can be notified. By way of example, if a detected respiration pattern crosses a threshold value or severity, an emergency responder can be notified. As another example, a respiratory pattern such as a Biot pattern or an ataxic pattern may indicate a serious injury or event. As such, in some embodiments, the system can notify an emergency responder if such a pattern is detected.

In some embodiments, devices or systems herein can take actions to address certain types of respiration patterns. For example, in some embodiments, if a hyperventilation respiration pattern is detected then the device or system can provide instructions to the device wearer on steps to take. For example, the device or system can provide breathing instructions that are paced sufficiently to bring the breathing pattern of the device wearer back to a normal breathing pattern. In some embodiments, the system can provide a suggestion or instruction to the device wearer to take a medication. In some embodiments, the system can provide a suggestion or instruction to the device wearer to sit down.

In various embodiments, ear-wearable systems can be configured so that respiration patterns are at least partially derived or confirmed from inputs provided by a device wearer. Such inputs can be direct inputs (e.g., an input that is directly related to respiration) or indirect inputs (e.g., an input that relates to or otherwise indicates a respiration pattern, but indirectly). As an example of a direct input, the ear-wearable system can be configured so that a device wearer input in the form of a “tap” of the device can signal that the device wearer is breathing in or out. In some embodiments, the ear-wearable system can be configured to generate a query for the device wearer and the device wearer input can be in the form of a response to the query.

Ear-wearable devices of the present disclosure can incorporate an antenna arrangement coupled to a high-frequency radio, such as a 2.4 GHz radio. The radio can conform to an IEEE 802.11 (e.g., WIFI®) or BLUETOOTH® (e.g., BLE, BLUETOOTH® 4. 2 or 5.0) specification, for example. It is understood that ear-wearable devices of the present disclosure can employ other radios, such as a 900 MHz radio or radios operating at other frequencies or frequency bands. Ear-wearable devices of the present disclosure can be configured to receive streaming audio (e.g., digital audio data or files) from an electronic or digital source. Representative electronic/digital sources (also referred to herein as accessory devices) include an assistive listening system, a TV streamer, a radio, a smartphone, a cell phone/entertainment device (CPED) or other electronic device that serves as a source of digital audio data or files. Systems herein can also include these types of accessory devices as well as other types of devices.

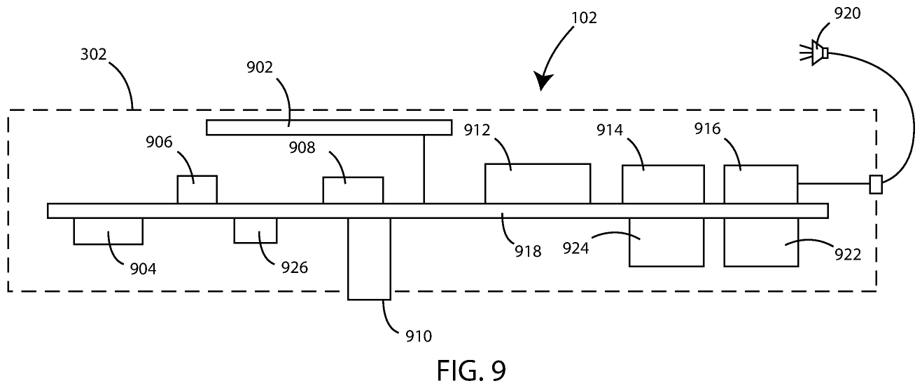

Referring now to FIG. 9, a schematic block diagram is shown with various components of an ear-wearable device in accordance with various embodiments. The block diagram of FIG. 9 represents a generic ear-wearable device for purposes of illustration. The ear-wearable device 102 shown in FIG. 9 includes several components electrically connected to a flexible mother circuit 918 (e.g., flexible mother board) which is disposed within housing 402. A power supply circuit 904 can include a battery and can be electrically connected to the flexible mother circuit 918 and provides power to the various components of the ear-wearable device 102. One or more microphones 906 are electrically connected to the flexible mother circuit 918, which provides electrical communication between the microphones 906 and a digital signal processor (DSP) 912. Microphones herein can be of various types including, but not limited to, unidirectional, omnidirectional, MEMS based microphones, piezoelectric microphones, magnetic microphones, electret condenser microphones, and the like. Among other components, the DSP 912 incorporates or is coupled to audio signal processing circuitry configured to implement various functions described herein. A sensor package 914 can be coupled to the DSP 912 via the flexible mother circuit 918. The sensor package 914 can include one or more different specific types of sensors such as those described in greater detail below. One or more user switches 910 (e.g., on/off, volume, mic directional settings) are electrically coupled to the DSP 912 via the flexible mother circuit 918. It will be appreciated that the user switches 910 can extend outside of the housing 402.

An audio output device 916 is electrically connected to the DSP 912 via the flexible mother circuit 918. In some embodiments, the audio output device 916 comprises a speaker (coupled to an amplifier). In other embodiments, the audio output device 916 comprises an amplifier coupled to an external receiver 920 adapted for positioning within an ear of a wearer. The external receiver 920 can include an electroacoustic transducer, speaker, or loud speaker. The ear-wearable device 102 may incorporate a communication device 908 coupled to the flexible mother circuit 918 and to an antenna 902 directly or indirectly via the flexible mother circuit 918. The communication device 908 can be a BLUETOOTH® transceiver, such as a BLE (BLUETOOTH® low energy) transceiver or other transceiver(s) (e.g., an IEEE 802.11 compliant device). The communication device 908 can be configured to communicate with one or more external devices, such as those discussed previously, in accordance with various embodiments. In various embodiments, the communication device 908 can be configured to communicate with an external visual display device such as a smart phone, a video display screen, a tablet, a computer, or the like.

In various embodiments, the ear-wearable device 102 can also include a control circuit 922 and a memory storage device 924. The control circuit 922 can be in electrical communication with other components of the device. In some embodiments, a clock circuit 926 can be in electrical communication with the control circuit. The control circuit 922 can execute various operations, such as those described herein. In various embodiments, the control circuit 922 can execute operations resulting in the provision of a user input interface by which the ear-wearable device 102 can receive inputs (including audible inputs, touch based inputs, and the like) from the device wearer. The control circuit 922 can include various components including, but not limited to, a microprocessor, a microcontroller, an FPGA (field-programmable gate array) processing device, an ASIC (application specific integrated circuit), or the like. The memory storage device 924 can include both volatile and non-volatile memory. The memory storage device 924 can include ROM, RAM, flash memory, EEPROM, SSD devices, NAND chips, and the like. The memory storage device 924 can be used to store data from sensors as described herein and/or processed data generated using data from sensors as described herein.

It will be appreciated that various of the components described in FIG. 9 can be associated with separate devices and/or accessory devices to the ear-wearable device. By way of example, microphones can be associated with separate devices and/or accessory devices. Similarly, audio output devices can be associated with separate devices and/or accessory devices to the ear-wearable device. Further accessory devices as discussed herein can include various of the components as described with respect to an ear-wearable device. For example, an accessory device can include a control circuit, a microphone, a motion sensor, and a power supply, amongst other things.

Accessory devices or external devices herein can include various different components. In some embodiments, the accessory device can be a personal communications device, such as a smart phone. However, the accessory device can also be other things such as a secondary wearable device, a handheld computing device, a dedicated location determining device (such as a handheld GPS unit), or the like.

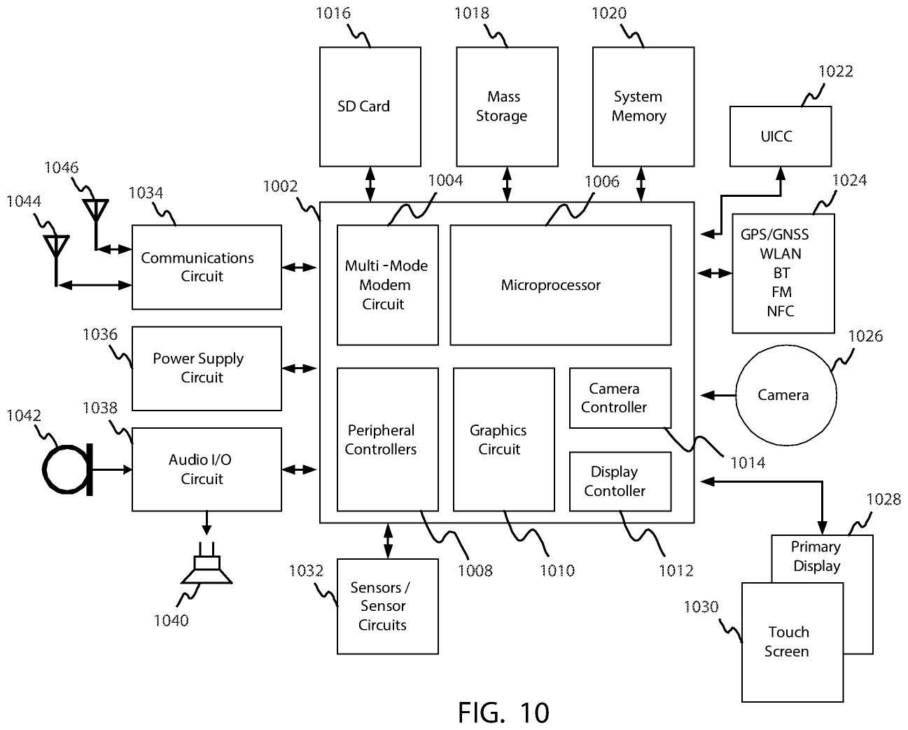

Referring now to FIG. 10, a schematic block diagram is shown of components of an accessory device (which could be a personal communications device or another type of accessory device) in accordance with various embodiments herein. This block diagram is just provided by way of illustration and it will be appreciated that accessory devices can include greater or lesser numbers of components. The accessory device in this example can include a control circuit 1002. The control circuit 1002 can include various components which may or may not be integrated. In various embodiments, the control circuit 1002 can include a microprocessor 1006, which could also be a microcontroller, FPGA, ASIC, or the like. The control circuit 1002 can also include a multi-mode modem circuit 1004 which can provide communications capability via various wired and wireless standards. The control circuit 1002 can include various peripheral controllers 1008. The control circuit 1002 can also include various sensors/sensor circuits 1032. The control circuit 1002 can also include a graphics circuit 1010, a camera controller 1014, and a display controller 1012. In various embodiments, the control circuit 1002 can interface with an SD card 1016, mass storage 1018, and system memory 1020. In various embodiments, the control circuit 1002 can interface with universal integrated circuit card (UICC) 1022. A spatial location determining circuit (or geolocation circuit) can be included and can take the form of an integrated circuit 1024 that can include components for receiving signals from GPS, GLONASS, BeiDou, Galileo, SBAS, WLAN, BT, FM, NFC type protocols, 5G picocells, or E911. In various embodiments, the accessory device can include a camera 1026. In various embodiments, the control circuit 1002 can interface with a primary display 1028 that can also include a touch screen 1030. In various embodiments, an audio I/O circuit 1038 can interface with the control circuit 1002 as well as a microphone 1042 and a speaker 1040. In various embodiments, a power supply or power supply circuit 1036 can interface with the control circuit 1002 and/or various other circuits herein in order to provide power to the system. In various embodiments, a communications circuit 1034 can be in communication with the control circuit 1002 as well as one or more antennas (1044, 1046).

It will be appreciated that in some cases a trend regarding respiration can be more important than an instantaneous measure or snapshot of respiration. For example, an hour-long trend where respiration rates rise to higher and higher levels may represent a greater health danger to an individual (and thus meriting intervention) than a brief spike in detected respiration rate. As such, in various embodiments herein the ear-wearable system is configured to record data regarding detected respiration and calculate a trend regarding the same. The trend can span minutes, hours, days, weeks, or months. Various actions can be taken by the system or device in response to the trend. For example, when the trend is adverse the device may initiate suggestions for corrective actions and/or increase the frequency with which such suggestions are provided to the device wearer. If suggestions are already being provided and/or actions are already being taken by the device and the trend is adverse the device may be configured to change the suggestions/instructions being provided to the device wearer as the current suggestions/instructions are being empirically shown to be ineffective.

### Feature Extraction

In various embodiments herein one or more microphones can be utilized to generate signals representative of sound. For example, in some embodiments, a front microphone can be used to generate signals representative of sound along with a rear microphone. The signals from the microphone(s) can be processed in order to evaluate/extract spectral and/or temporal features therefrom. Many different spectral and/or temporal features can be evaluated/extracted including, but not limited to, those shown in the following table.

TABLE 1
Feature Name

Zero-Crossing Rate
Periodicity Strength
Short Time Energy
Spectral Centroid
Spectral Centroid Mean
Spectral Bandwidth
Spectral Roll-off
Spectral Flux
High-/Low-Frequency Energy Ratio
High-Frequency Slope
Low-Frequency Slope
Absolute Magnitude Difference Function
Spectral Flux at High Frequency
Spectral Flux at Low Frequency
Periodicity Strength
Low Frequency Envelope Peakiness
Onset Rate

Spectral and/or temporal features that can be utilized from signals of a single-mic can include, but are not limited to, HLF (the relative power in the high-frequency portion of the spectrum relative to the low-frequency portion), SC (spectral centroid), LS (the slope of the power spectrum below the Spectral Centroid), PS (periodic strength), and Envelope Peakiness (a measure of signal envelope modulation).

In embodiments with at least two microphones, one or more of the following signal features can be used to detect respiration phases or events using the spatial information between two microphones.
MSC: Magnitude Squared Coherence.
ILD: level difference
IPD: phase difference

The MSC feature can be used to determine whether a source is a point source or distributed. The ILD and IPD features can be used to determine the direction of arrival of the sound. Breathing sounds are generally located at a particular location relative to the microphones on the device. Also breathing sounds are distributed in spatial origin in contrast to speech which is mostly emitted from the lips.

It will be appreciated that when at least two microphones are used that have some physical separation from one another that the signals can then be processed to derive/extract/utilize spatial information. For example, signals from a front microphone and a rear microphone can be correlated in order to extract those signals representing sound with a point of origin falling in an area associated with the inside of the device wearer. As such, this operation can be used to separate signals associated with external noise and external speech from signals associated with breathing sounds of the device wearer.

Using data associated with the sensor signals directly, spectral features of the sensor signals, and/or data associated with spatial features, an operation can be executed in order to detect respiration, respiration phases, respiration events, and the like.

### Pattern Identification

It will be appreciated that in various embodiments herein, a device or a system can be used to detect a pattern or patterns indicative of respiration, respiration events, a respiration pattern, a respiration condition, or the like. Such patterns can be detected in various ways. Some techniques are described elsewhere herein, but some further examples will now be described.

As merely one example, one or more sensors can be operatively connected to a controller (such as the control circuit described in FIG. 10) or another processing resource (such as a processor of another device or a processing resource in the cloud). The controller or other processing resource can be adapted to receive data representative of a characteristic of the subject from one or more of the sensors and/or determine statistics of the subject over a monitoring time period based upon the data received from the sensor. As used herein, the term “data” can include a single datum or a plurality of data values or statistics. The term “statistics” can include any appropriate mathematical calculation or metric relative to data interpretation, e.g., probability, confidence interval, distribution, range, or the like. Further, as used herein, the term “monitoring time period” means a period of time over which characteristics of the subject are measured and statistics are determined. The monitoring time period can be any suitable length of time, e.g., 1 millisecond, 1 second, 10 seconds, 30 seconds, 1 minute, 10 minutes, 30 minutes, 1 hour, etc., or a range of time between any of the foregoing time periods.

Any suitable technique or techniques can be utilized to determine statistics for the various data from the sensors, e.g., direct statistical analyses of time series data from the sensors, differential statistics, comparisons to baseline or statistical models of similar data, etc. Such techniques can be general or individual-specific and represent long-term or short-term behavior. These techniques could include standard pattern classification methods such as Gaussian mixture models, clustering as well as Bayesian approaches, machine learning approaches such as neural network models and deep learning, and the like.

Further, in some embodiments, the controller can be adapted to compare data, data features, and/or statistics against various other patterns, which could be prerecorded patterns (baseline patterns) of the particular individual wearing an ear-wearable device herein, prerecorded patterns (group baseline patterns) of a group of individuals wearing ear-wearable devices herein, one or more predetermined patterns that serve as patterns indicative of indicative of an occurrence of respiration or components thereof such as inspiration, expiration, respiration sounds, and the like (positive example patterns), one or more predetermined patterns that serve as patterns indicative of the absence of such things (negative example patterns), or the like. As merely one scenario, if a pattern is detected in an individual that exhibits similarity crossing a threshold value to a particular positive example pattern or substantial similarity to that pattern, wherein the pattern is specific for a respiration event or phase, a respiration pattern, a particular type of respiration sound, or the like, then that can be taken as an indication of an occurrence of that type of event experienced by the device wearer.

Similarity and dissimilarity can be measured directly via standard statistical metrics such normalized Z-score, or similar multidimensional distance measures (e.g., Mahalanobis or Bhattacharyya distance metrics), or through similarities of modeled data and machine learning. These techniques can include standard pattern classification methods such as Gaussian mixture models, clustering as well as Bayesian approaches, neural network models, and deep learning.

As used herein the term “substantially similar” means that, upon comparison, the sensor data are congruent or have statistics fitting the same statistical model, each with an acceptable degree of confidence. The threshold for the acceptability of a confidence statistic may vary depending upon the subject, sensor, sensor arrangement, type of data, context, condition, etc.

The statistics associated with the health status of an individual (and, in particular, their status with respect to respiration), over the monitoring time period, can be determined by utilizing any suitable technique or techniques, e.g., standard pattern classification methods such as Gaussian mixture models, clustering, hidden Markov models, as well as Bayesian approaches, neural network models, and deep learning.

Various embodiments herein specifically include the application of a machine learning classification model. In various embodiments, the ear-wearable system can be configured to periodically update the machine learning classification model based on indicators of respiration of the device wearer.

In some embodiments, a training set of data can be used in order to generate a machine learning classification model. The input data can include microphone and/or sensor data as described herein as tagged/labeled with binary and/or non-binary classifications of respiration, respiration events or phases, respiration patterns, respiratory conditions, or the like. Binary classification approaches can utilize techniques including, but not limited to, logistic regression, k-nearest neighbors, decision trees, support vector machine approaches, naive Bayes techniques, and the like. In some embodiments herein, a multi-node decision tree can be used to reach a binary result (e.g. binary classification) on whether the individual is breathing or not, inhaling or not, exhaling or not, and the like.

In some embodiments, signals or other data derived therefrom can be divided up into discrete time units (such as periods of milliseconds, seconds, minutes, or longer) and the system can perform binary classification (e.g., “inhaling” or “not inhaling”) regarding whether the individual was inhaling (or any other respiration event) during that discrete time unit. As an example, in some embodiments, signal processing or evaluation operations herein to identify respiratory events can include binary classification on a per second (or different time scale) basis.

Multi-class classification approaches (e.g., for non-binary classifications of respiration, respiration events or phases, respiration patterns, respiratory conditions, or the like) can include k-nearest neighbors, decision trees, naive Bayes approaches, random forest approaches, and gradient boosting approaches amongst others.

In various embodiments, the ear-wearable system is configured to execute operations to generate or update the machine learning model on the ear-wearable device itself. In some embodiments, the ear-wearable system may convey data to another device such as an accessory device or a cloud computing resource in order to execute operations to generate or update a machine learning model herein. In various embodiments, the ear-wearable system is configured to weight certain possible markers of respiration in the machine learning classification model more heavily based on derived correlations specific for the individual as described elsewhere herein.

In addition to or in replacement of the application of machine learning models, in some embodiments signal processing techniques (such as a matched filter approach) can be applied to analyze sensor signals and detect a respiratory condition and/or parameter based on analysis of the signals. In a matched filter approach, the system can correlate a known signal, or template (such as a template serving as an example of a particular type of respiration parameter, pattern, or condition), with sensor signals to detect the presence of the template in the sensor signals. This is equivalent to convolving the sensor signal with a conjugated time-reversed version of the template.

### Correlated Factors/Data

In some cases, other types of data can also be evaluated when identifying a respiratory event. For example, sounds associated with breathing can be different depending on whether the device wearer is sitting, standing, or lying down. Thus, in some embodiments herein the ear-wearable device or system can be configured to evaluate the signals from a motion sensor (which can include an accelerometer, gyroscope, or the like) or other sensor to identify the device wearer's posture. For example, the process of sitting down includes a characteristic motion pattern that can be identified from evaluation of a motion sensor signal. Weighting factors for identification of a respiration event can be adjusted if the system detects that the individual has assumed a specific posture. In some embodiments, a different machine learning classification model can be applied depending on the posture of the device wearer.

Physical exertion can drive changes in respiration including increasing respiration rate. As such, in can be important to consider markers of physical exertion when evaluating signals from sensors and/or microphones herein to detect respiration patterns and/or respiration events. In some embodiments, the device or system can evaluate signals from a motion sensor to detect motion that is characteristic of exercise such as changes in an accelerometer signal consistent with foot falls as a part of walking or running. Weighting factors for identification of a respiration event can be adjusted if the system detects that the individual is physically exerting themselves. In some embodiments, a different machine learning classification model can be applied depending on the physical exertion level of the device wearer.

In some scenarios, factors such as the time of the year may impact a device wearer and their breathing sounds. For example, pollen may be present in specific geolocations in greater amounts at certain times of the year. The pollen can trigger allergies in the device wearer which, in turn, can influence breathing sounds of the individual. Thus, in various embodiments herein the device and/or system can also evaluate the time of the year when evaluating microphone and/or sensor signals to detect respiration events. For example, weighting factors for identification of a respiration event can be adjusted based on the time of year. In some embodiments, a different machine learning classification model can be applied depending on the current time of year.

In some scenarios, factors such as geolocation may impact a device wearer and their breathing sounds. Geolocation can be determined via a geolocation circuit as described herein. For example, conditions may be present in specific geolocations that can influence detected breathing sounds of the individual. As another example, certain types of infectious disease impacting respiration may be more common at a specific geolocation. Thus, in various embodiments herein the device and/or system can also the current geolocation of the device wearer when evaluating microphone and/or sensor signals to detect respiration events. For example, weighting factors for identification of a respiration event can be adjusted based on the current geolocation. In some embodiments, a different machine learning classification model can be applied depending on the current geolocation of the device wearer.

### Sensor Package

Various embodiments herein include a sensor package. Specifically, systems and ear-wearable devices herein can include one or more sensor packages (including one or more discrete or integrated sensors) to provide data for use with operations to respiration of an individual. Further details about the sensor package are provided as follows. However, it will be appreciated that this is merely provided by way of example and that further variations are contemplated herein. Also, it will be appreciated that a single sensor may provide more than one type of physiological data. For example, heart rate, respiration, blood pressure, or any combination thereof may be extracted from PPG sensor data.

In various embodiments, detection of aspects related to respiration is detected from analysis of data produced by at least one of the microphone and the sensor package. In various embodiments, the sensor package can include at least one including at least one of a heart rate sensor, a heart rate variability sensor, an electrocardiogram (ECG) sensor, a blood oxygen sensor, a blood pressure sensor, a skin conductance sensor, a photoplethysmography (PPG) sensor, a temperature sensor (such as a core body temperature sensor, skin temperature sensor, ear-canal temperature sensor, or another temperature sensor), a motion sensor, an electroencephalograph (EEG) sensor, and a respiratory sensor. In various embodiments, the motion sensor can include at least one of an accelerometer and a gyroscope.

The sensor package can comprise one or a multiplicity of sensors. In some embodiments, the sensor packages can include one or more motion sensors (or movement sensors) amongst other types of sensors. Motion sensors herein can include inertial measurement units (IMU), accelerometers, gyroscopes, barometers, altimeters, and the like. The IMU can be of a type disclosed in commonly owned U.S. patent application Ser. No. 15/331,230, filed Oct. 21, 2016, which is incorporated herein by reference. In some embodiments, electromagnetic communication radios or electromagnetic field sensors (e.g., telecoil, NFMI, TMR, GMR, etc.) sensors may be used to detect motion or changes in position. In some embodiments, biometric sensors may be used to detect body motions or physical activity. Motions sensors can be used to track movements of a device wearer in accordance with various embodiments herein.

In some embodiments, the motion sensors can be disposed in a fixed position with respect to the head of a device wearer, such as worn on or near the head or ears. In some embodiments, the operatively connected motion sensors can be worn on or near another part of the body such as on a wrist, arm, or leg of the device wearer.

According to various embodiments, the sensor package can include one or more of an IMU, and accelerometer (3, 6, or 9 axis), a gyroscope, a barometer (or barometric pressure sensor), an altimeter, a magnetometer, a magnetic sensor, an eye movement sensor, a pressure sensor, an acoustic sensor, a telecoil, a heart rate sensor, a global positioning system (GPS), a temperature sensor, a blood pressure sensor, an oxygen saturation sensor, an optical sensor, a blood glucose sensor (optical or otherwise), a galvanic skin response sensor, a histamine level sensor (optical or otherwise), a microphone, acoustic sensor, an electrocardiogram (ECG) sensor, electroencephalography (EEG) sensor which can be a neurological sensor, a sympathetic nervous stimulation sensor (which in some embodiments can including other sensors described herein to detect one or more of increased mental activity, increased heart rate and blood pressure, an increase in body temperature, increased breathing rate, or the like), eye movement sensor (e.g., electrooculogram (EOG) sensor), myographic potential electrode sensor (or electromyography—EMG), a heart rate monitor, a pulse oximeter or oxygen saturation sensor (SpO2), a wireless radio antenna, blood perfusion sensor, hydrometer, sweat sensor, cerumen sensor, air quality sensor, pupillometry sensor, cortisol level sensor, hematocrit sensor, light sensor, image sensor, and the like.

In some embodiments herein, the ear-wearable device or system can include an air quality sensor. In some embodiments herein, the ear-wearable device or system can include a volatile organic compounds (VOCs) sensor. In some embodiments, the ear-wearable device or system can include a particulate matter sensor.

In lieu of, or in addition to, sensors for certain properties as described herein, the same information can be obtained via interface with another device and/or through an API as accessed via a data network using standard techniques for requesting and receiving information.

In some embodiments, the sensor package can be part of an ear-wearable device. However, in some embodiments, the sensor packages can include one or more additional sensors that are external to an ear-wearable device. For example, various of the sensors described above can be part of a wrist-worn or ankle-worn sensor package, or a sensor package supported by a chest strap. In some embodiments, sensors herein can be disposable sensors that are adhered to the device wearer (“adhesive sensors”) and that provide data to the ear-wearable device or another component of the system.

Data produced by the sensor(s) of the sensor package can be operated on by a processor of the device or system.

As used herein the term “inertial measurement unit” or “IMU” shall refer to an electronic device that can generate signals related to a body's specific force and/or angular rate. IMUs herein can include one or more accelerometers (3, 6, or 9 axis) to detect linear acceleration and a gyroscope to detect rotational rate. In some embodiments, an IMU can also include a magnetometer to detect a magnetic field.

The eye movement sensor may be, for example, an electrooculographic (EOG) sensor, such as an EOG sensor disclosed in commonly owned U.S. Pat. No. 9,167,356, which is incorporated herein by reference. The pressure sensor can be, for example, a MEMS-based pressure sensor, a piezo-resistive pressure sensor, a flexion sensor, a strain sensor, a diaphragm-type sensor, and the like.

The temperature sensor can be, for example, a thermistor (thermally sensitive resistor), a resistance temperature detector, a thermocouple, a semiconductor-based sensor, an infrared sensor, or the like.

The blood pressure sensor can be, for example, a pressure sensor. The heart rate sensor can be, for example, an electrical signal sensor, an acoustic sensor, a pressure sensor, an infrared sensor, an optical sensor, or the like.

The electrical signal sensor can include two or more electrodes and can include circuitry to sense and record electrical signals including sensed electrical potentials and the magnitude thereof (according to Ohm's law where V=IR) as well as measure impedance from an applied electrical potential. The electrical signal sensor can be an impedance sensor.

The oxygen saturation sensor (such as a blood oximetry sensor) can be, for example, an optical sensor, an infrared sensor, a visible light sensor, or the like.

It will be appreciated that the sensor package can include one or more sensors that are external to the ear-wearable device. In addition to the external sensors discussed hereinabove, the sensor package can comprise a network of body sensors (such as those listed above) that sense movement of a multiplicity of body parts (e.g., arms, legs, torso). In some embodiments, the ear-wearable device can be in electronic communication with the sensors or processor of another medical device, e.g., an insulin pump device or a heart pacemaker device.

In various embodiments herein, a device or system can specifically include an inward-facing microphone (e.g., facing the ear canal, or facing tissue, as opposed to facing the ambient environment.) A sound signal captured by the inward-facing microphone can be used to determine physiological information, such as sounds relating to respiration or another property of interest. For example, a signal from an inward-facing microphone may be used to determine heart rate, respiration, or both, e.g., from sounds transferred through the body. In some examples, a measure of blood pressure may be determined, e.g., based on an amplitude of a detected physiologic sound (e.g., louder sound correlates with higher blood pressure.)

### Methods

Many different methods are contemplated herein, including, but not limited to, methods of making devices, methods of using devices, methods of detecting aspects related to respiration, methods of monitoring aspects related to respiration, and the like. Aspects of system/device operation described elsewhere herein can be performed as operations of one or more methods in accordance with various embodiments herein.

In an embodiment, a method of detecting respiratory conditions and/or parameters with an ear-wearable device is included, the method including analyzing signals from a microphone and/or a sensor package and detecting a respiratory condition and/or parameter based on analysis of the signals.

In an embodiment, the method can further include operating the ear-wearable device in a onset detection mode and operating the ear-wearable device in an event classification mode when the onset of an event is detected.

In an embodiment, the method can further include buffering signals from the microphone and/or the sensor package, executing a feature extraction operation, and classifying the event when operating in the event classification mode.

In an embodiment, the method can further include operating in a setup mode prior to operating in the onset detection mode and the event classification mode.

In an embodiment, the method can further include querying a device wearer to take a respiratory action when operating in the setup mode. In an embodiment, the method can further include querying a device wearer to reproduce a respiratory event when operating in the setup mode.

In an embodiment, the method can further include receiving and executing a machine learning classification model specific for the detection of one or more respiratory conditions. In an embodiment, the method can further include receiving and executing a machine learning classification model that is specific for the detection of one or more respiratory conditions that are selected based on a user input from amongst a set of respiratory conditions.

In an embodiment, the method can further include sending information regarding detected respiratory conditions and/or parameters to an accessory device for presentation to the device wearer.

In an embodiment, the method can further include detecting one or more adventitious sounds. In an embodiment, the adventitious sounds can include at least one selected from the group consisting of fine crackles, medium crackles, coarse crackles, wheezing, rhonchi, and pleural friction rub.

In an embodiment, a method of detecting respiratory conditions and/or parameters with an ear-wearable device system is included. The method can include analyzing signals from a microphone and/or a sensor package with an ear-wearable device, detecting the onset of a respiratory event with the ear-wearable device, buffering signals from the microphone and/or the sensor package after a detected onset, sending buffered signal data from the ear-wearable device to an accessory device, processing signal data from the ear-wearable device with the accessory device to detect a respiratory condition, and sending an indication of a respiratory condition from the accessory device to the ear-wearable device.

It should be noted that, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as arranged and configured, constructed and arranged, constructed, manufactured and arranged, and the like.

All publications and patent applications in this specification are indicative of the level of ordinary skill in the art to which this invention pertains. All publications and patent applications are herein incorporated by reference to the same extent as if each individual publication or patent application was specifically and individually indicated by reference.

As used herein, the recitation of numerical ranges by endpoints shall include all numbers subsumed within that range (e.g., 2 to 8 includes 2.1, 2.8, 5.3, 7, etc.).

The headings used herein are provided for consistency with suggestions under 37 CFR 1.77 or otherwise to provide organizational cues. These headings shall not be viewed to limit or characterize the invention(s) set out in any claims that may issue from this disclosure. As an example, although the headings refer to a “Field,” such claims should not be limited by the language chosen under this heading to describe the so-called technical field. Further, a description of a technology in the “Background” is not an admission that technology is prior art to any invention(s) in this disclosure. Neither is the “Summary” to be considered as a characterization of the invention(s) set forth in issued claims.

The embodiments described herein are not intended to be exhaustive or to limit the invention to the precise forms disclosed in the following detailed description. Rather, the embodiments are chosen and described so that others skilled in the art can appreciate and understand the principles and practices. As such, aspects have been described with reference to various specific and preferred embodiments and techniques. However, it should be understood that many variations and modifications may be made while remaining within the spirit and scope herein.

1. An ear-wearable device for respiratory monitoring comprising:
a control circuit;
a microphone, wherein the microphone is in electrical communication with the control circuit; and
a sensor package, wherein the sensor package is in electrical communication with the control circuit;
wherein the ear-wearable device for respiratory monitoring is configured to
analyze signals from the microphone and/or the sensor package; and
detect a respiratory condition and/or parameter based on analysis of the signals.

2. The ear-wearable device for respiratory monitoring of claim 1, wherein the ear-wearable device for respiratory monitoring is configured to
operate in an onset detection mode; and
operate in an event classification mode when the onset of an event is detected.

3. The ear-wearable device for respiratory monitoring of claim 2, wherein the ear-wearable device for respiratory monitoring is configured to buffer signals from the microphone and/or the sensor package, execute a feature extraction operation, and classify the event when operating in the event classification mode.

4. The ear-wearable device for respiratory monitoring of claim 2, wherein the ear-wearable device for respiratory monitoring is configured to operate in a setup mode prior to operating in the onset detection mode and the event classification mode.

5. The ear-wearable device for respiratory monitoring of claim 4, wherein the ear-wearable device for respiratory monitoring is configured to query a device wearer to take a respiratory action when operating in the setup mode.

6. The ear-wearable device for respiratory monitoring of claim 4, wherein the ear-wearable device for respiratory monitoring is configured to query a device wearer to reproduce a respiratory event when operating in the setup mode.

7. (canceled)

8. The ear-wearable device for respiratory monitoring of claim 1, wherein the ear-wearable device for respiratory monitoring is configured to receive and execute a machine learning classification model that is specific for the detection of one or more respiratory conditions that are selected based on a user input from amongst a set of respiratory conditions.

9. The ear-wearable device for respiratory monitoring of claim 1, wherein the ear-wearable device for respiratory monitoring is configured to send information regarding detected respiratory conditions and/or parameters to an accessory device for presentation to the device wearer.

10. The ear-wearable device for respiratory monitoring of claim 1, the respiratory condition and/or parameter comprising at least one selected from the group consisting of respiration rate, tidal volume, respiratory minute volume, inspiratory reserve volume, expiratory reserve volume, vital capacity, and inspiratory capacity.

11. The ear-wearable device for respiratory monitoring of claim 1, the respiratory condition and/or parameter comprising at least one selected from the group consisting of bradypnea, tachypnea, hyperpnea, an obstructive respiration condition, Kussmaul respiration, Biot respiration, ataxic respiration, and Cheyne-Stokes respiration.

12. The ear-wearable device for respiratory monitoring of claim 1, wherein the ear-wearable device for respiratory monitoring is configured to detect one or more adventitious sounds.

13. (canceled)

14. An ear-wearable system for respiratory monitoring comprising:
an accessory device, the accessory device comprising
a first control circuit; and
a display screen;
an ear-wearable device, the ear-wearable device comprising
a second control circuit;
a microphone, wherein the microphone is in electrical communication with the second control circuit; and
a sensor package, wherein the sensor package is in electrical communication with the second control circuit;
wherein the ear-wearable device is configured to
analyze signals from the microphone and/or the sensor package to detect the onset of a respiratory event and buffer signals from the microphone and/or the sensor package after a detected onset;
send buffered signal data to the accessory device; and
receive an indication of a respiratory condition from the accessory device; and
wherein the accessory device is configured to process signal data from the ear-wearable device to detect a respiratory condition.

15. The ear-wearable system for respiratory monitoring of claim 14, wherein the ear-wearable system for respiratory monitoring is configured to
operate in a onset detection mode; and
operate in an event classification mode when the onset of an event is detected.

16. The ear-wearable system for respiratory monitoring of claim 15, wherein the ear-wearable device is configured to buffer signals from the microphone and/or the sensor package when operating in the event classification mode.

17. The ear-wearable system for respiratory monitoring of claim 15, wherein the ear-wearable system for respiratory monitoring is configured to operate in a setup mode prior to operating in the onset detection mode and the event classification mode.

18. The ear-wearable system for respiratory monitoring of claim 17, wherein the ear-wearable system for respiratory monitoring is configured to query a device wearer to take a respiratory action when operating in the setup mode.

19. The ear-wearable system for respiratory monitoring of claim 17, wherein the ear-wearable system for respiratory monitoring is configured to query a device wearer to reproduce a respiratory event when operating in the setup mode.

20. (canceled)

21. The ear-wearable system for respiratory monitoring of claim 14, wherein the ear-wearable system for respiratory monitoring is configured to receive and execute a machine learning classification model that is specific for the detection of one or more respiratory conditions that are selected based on a user input from amongst a set of respiratory conditions.

22. The ear-wearable system for respiratory monitoring of claim 14, wherein the accessory device is configured to present information regarding detected respiratory conditions and/or parameters to the device wearer.

23. The ear-wearable system for respiratory monitoring of claim 14, the respiratory condition comprising at least one selected from the group consisting of bradypnea, tachypnea, hyperpnea, an obstructive respiration condition, Kussmaul respiration, Biot respiration, ataxic respiration, and Cheyne-Stokes respiration.

24-47. (canceled)
